(19) United States
(12) Patent Application Publication
(10) Pub. No.: US 2021/0315887 A1
(43) Pub. Date: Oct. 14, 2021

(54) METHODS OF ADMINISTERING THERAPY

(71) Applicant: GALAPAGOS NV, Mechelen (BE)

(72) Inventors: Julie DESRIVOT, Toulouse (FR); Florence Sylvie NAMOUR, Romainville (FR)

(21) Appl. No.: 17/266,361

(22) PCT Filed: Aug. 7, 2019
(86) PCT No.: PCT/EP2019/071170

§ 371 Date: Feb. 5, 2021

(30) Foreign Application Priority Data
Aug. 8, 2018 (GB) 1812861.1

(51) Int. Cl. A61K 31/496

(52) U.S. Cl. CPC A61K 31/496

(57) ABSTRACT

The present invention relates to improved methods of administering compound 1 therapy, involving increased effectiveness of compound 1 by avoiding or contraindicating combined CYP3A4/P-gp inducers, e.g. rifampicin.

The present invention relates to improved methods of administering compound 1 therapy, involving increased effectiveness of compound 1 by avoiding or contraindicating combined CYP3A4/P-gp inducers, e.g. rifampicin.

### BACKGROUND

Compound 1 is a small molecule drug under investigation having structural formula I.

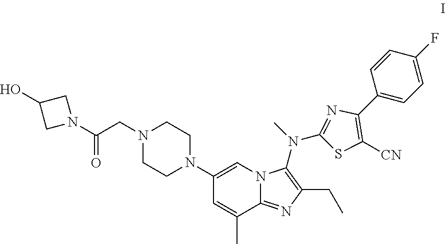 [SMILES: CCc1nc2c(C)cc(N3CCN(CC(=O)N4CC(O)C4)CC3)cn2c1N(C)c1nc(-c2ccc(F)cc2)c(C#N)s1]

The structure, name and synthesis are known and described in WO 2014/139882. Compound 1 is being developed as an autotaxin inhibitor and studied for the treatment of pulmonary fibrosis, and idiopathic pulmonary fibrosis in particular (Maher T M et al. “Safety, tolerability, pharmacokinetics, and pharmacodynamics of GLPG1690, a novel autotaxin inhibitor, to treat idiopathic pulmonary fibrosis (FLORA): a phase 2a randomised placebo-controlled trial” The Lancet Respiratory Medicine May 2018).

Rifampicin is an antibiotic used to treat bacterial infections with a particular role in the treatment of tuberculosis (TB).

### SUMMARY OF THE INVENTION

The invention described herein is based upon the observation of reduced systemic exposure of compound 1 in patients who are also administered rifampicin.

Accordingly, in a first aspect the present invention provides Compound 1 for use in treating a patient in need of compound 1 therapy, characterized in that the treating comprises avoiding, contraindicating or discontinuing concomitant use or co-administration of a combined CYP3A4/P-gp inducer.

In a second aspect, the present invention also provides the use of compound 1 in the manufacture of a medicament for treating a patient in need of therapy using compound 1, characterized in that the treating comprises avoiding, contraindicating or discontinuing concomitant use or co-administration of a combined CYP3A4/P-gp inducer.

In a further aspect, the present invention provides a method of administering treatment using compound 1 to a patient in need of therapy using compound 1 comprising administering the patient a therapeutically effective amount of compound 1, and, avoiding, contraindicating or discontinuing concomitant use or co-administration of a combined CYP3A4/P-gp inducer.

In specific embodiments, the combined CYP3A4/P-gp inducer is rifampicin. Further embodiments are apparent from the detailed description herein below.

### FIGURES

FIG. 1: Mean (±SE) compound 1 Plasma Concentrations (ng/mL) over Time (Log-Linear Scale): solid line represent data obtained on day 1, dotted line represents data obtained on day 12.

### DETAILED DESCRIPTION

Compound 1 is a small molecule autotaxin (ATX) inhibitor having structural formula I.

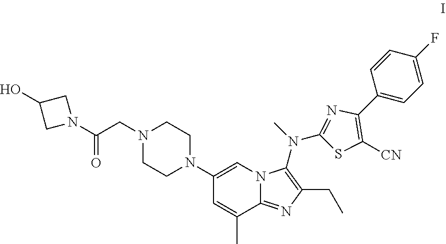 [SMILES: CCc1nc2c(C)cc(N3CCN(CC(=O)N4CC(O)C4)CC3)cn2c1N(C)c1nc(-c2ccc(F)cc2)c(C#N)s1]

The structure, name and synthesis are known and described in WO 2014/139882. Compound 1 is being investigated as an orally active agent for medical use in human.

As used herein, a patient “in need of compound 1 therapy” or “in need of compound 1 treatment” is a patient who would benefit from administration of compound 1. Such patient may be suffering from any disease or condition for which compound 1 treatment may be useful in ameliorating symptoms. In embodiments, a patient in need of compound 1 therapy or treatment is a patient suffering disease or having a condition that benefits autotaxin inhibition. Such diseases or conditions include fibrotic diseases, proliferative diseases, inflammatory diseases, autoimmune diseases, respiratory diseases, cardiovascular diseases, neurodegenerative diseases, dermatological disorders, and/or abnormal angiogenesis associated diseases.

In embodiments, a patient in need of compound 1 therapy or treatment is a patient suffering fibrotic disease. In a particular embodiment, the fibrotic disease is selected from idiopathic pulmonary fibrosis (IPF), cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease), radiation induced fibrosis, chronic obstructive pulmonary disease (COPD), scleroderma, bleomycin induced pulmonary fibrosis, chronic asthma, silicosis, asbestos induced pulmonary fibrosis, acute respiratory distress syndrome (ARDS), kidney fibrosis, tubulointerstitium fibrosis, glomerular nephritis, focal segmental glomerular sclerosis, IgA nephropathy, hypertension, Alport, gut fibrosis, liver fibrosis, cirrhosis, alcohol induced liver fibrosis, toxic/drug induced liver fibrosis, hemochromatosis, nonalcoholic steatohepatitis (NASH), biliary duct injury, primary biliary cirrhosis, infection induced liver fibrosis, viral induced liver fibrosis, and autoimmune hepatitis, corneal scarring, hypertrophic scarring, Dupuytren disease, keloids, cutaneous fibrosis, cutaneous scleroderma, systemic sclerosis, spinal cord injury/fibrosis, myelofibrosis, vascular restenosis, atherosclerosis, arteriosclerosis, Wegener's granulomatosis, Peyronie's disease, or chronic lymphocytic. More particularly, the fibrotic diseases is idiopathic pulmonary fibrosis (IPF).

In embodiments, a patient in need of compound 1 therapy or treatment is a patient suffering proliferative disease. In a particular embodiment, the proliferative disease is selected from cancer, leukemia, multiple myeloma and psoriasis.

In embodiments, a patient in need of compound 1 therapy or treatment is a patient suffering inflammatory diseases. In a particular embodiment, the inflammatory disease is selected from rheumatoid arthritis, osteoarthritis, allergic airway disease (e.g. asthma), chronic obstructive pulmonary disease (COPD) and inflammatory bowel diseases (e.g. Crohn's disease and ulcerative colitis). More particularly, the inflammatory disease is selected from rheumatoid arthritis, and chronic obstructive pulmonary disease (COPD).

In embodiments, a patient in need of compound 1 therapy or treatment is a patient suffering autoimmune diseases. In a particular embodiment, the autoimmune disease is selected from COPD, asthma (e.g intrinsic asthma, extrinsic asthma, dust asthma, infantily asthma) particularly chronic or inveterate asthma (for example late asthma and airway hyperreponsiveness), bronchitis, including bronchial asthma, systemic lupus erythematosus (SLE), cutaneous lupus erythrematosis, lupus nephritis, dermatomyositis, Sjogren's syndrome, multiple sclerosis, psoriasis, dry eye disease, type I diabetes mellitus and complications associated therewith, atopic eczema (atopic dermatitis), thyroiditis (Hashimoto's and autoimmune thyroiditis), contact dermatitis and further eczematous dermatitis, inflammatory bowel disease (e.g. Crohn's disease and ulcerative colitis), atherosclerosis and amyotrophic lateral sclerosis. Particularly, the autoimmune disease is selected from COPD, asthma, systemic lupus erythematosis, type I diabetes mellitus and inflammatory bowel disease.

In a specific embodiment, a patient in need of compound 1 therapy or treatment is a patient suffering a respiratory disease. In a particular embodiment, the respiratory disease is selected from asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia.

In a specific embodiment, a patient in need of compound 1 therapy or treatment is a patient suffering cardiovascular diseases. In a particular embodiment, the cardiovascular disease is selected from arrhythmia (atrial or ventricular or both), atherosclerosis and its sequelae, angina, cardiac rhythm disturbances, myocardial ischemia, myocardial infarction, cardiac or vascular aneurysm, vasculitis, stroke, peripheral obstructive arteriopathy of a limb, an organ, or a tissue, reperfusion injury following ischemia of the brain, heart, kidney or other organ or tissue, endotoxic, surgical, or traumatic shock, hypertension, valvular heart disease, heart failure, abnormal blood pressure, shock, vasoconstriction (including that associated with migraines), vascular abnormality, inflammation, insufficiency limited to a single organ or tissue.

In a specific embodiment, a patient in need of compound 1 therapy or treatment is a patient suffering neurodegenerative diseases. In a particular embodiment, the neurodegenerative disease is selected from Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis (ALS or Lou Gehrig's Disease), Huntington's disease, and prion diseases.

In a specific embodiment, a patient in need of compound 1 therapy or treatment is a patient suffering dermatological disorders. In a particular embodiment, the dermatological disease is selected from atopic dermatitis, bullous disorders, collagenoses, psoriasis, psoriatic lesions, dermatitis, contact dermatitis, eczema, pruritus, urticaria, rosacea, scleroderma, wound healing, scarring, hypertrophic scarring, keloids, Kawasaki Disease, rosacea, or Sjogren-Larsso Syndrome. In a particular embodiment, a patient in need of compound 1 therapy or treatment is a patient suffering from scleroderma.

In a specific embodiment, a patient in need of compound 1 therapy or treatment is a patient suffering abnormal angiogenesis associated diseases. In a particular embodiment, the abnormal angiogenesis associated disease is selected from atherosclerosis, hypertension, tumor growth, inflammation, rheumatoid arthritis, wet-form macular degeneration, choroidal neovascularization, retinal neovascularization, diabetic retinopathy, and glioblastoma multiforma.

Cytochrome P450 3A4, also referred to by its gene name “CYP3A4”, is a member of the cytochrome P450 family of monooxygenases. The CYP3A4 protein localizes to the endoplasmic reticulum and is expressed primarily in the liver and the intestines, and is involved in metabolization of selected drugs.

P-glycoprotein (P-gp) is also referred to as “Multidrug Resistance Protein (MDR1)” and by its gene name “ABCB1”. P-glycoprotein is a member of a class of transport molecules called “ATP Binding Cassette” transporters or “ABC” transporters for short. P-glycoprotein is located in the cell membrane of various tissues, such as in the intestines, kidney, liver and at the blood-brain barrier. P-glycoprotein transports substances such as a drug administered to the individual (unchanged or after they are metabolized) out of a cell (efflux) so that they can be eliminated from the body.

A combined CYP3A4/P-gp inducer is a substance that upon administration to a subject increases CYP3A4 and P-gp mediated activity. A combined CYP3A4/P-gp inducer that decreases the availability of compound 1 may be selected from the group comprising phenytoin, carbamazepine, rifampicin, dexamethasone and St John's wort. In a particular embodiment the CYP3A4/P-gp inducer is selected from phenytoin, carbamazepine and rifampicin. In a specific embodiment, the CYP3A4/P-gp inducer is rifampicin.

Rifampicin (also known as rifampin or rifadin) is an antibiotic. “Rifampicin indications” (prophylactic and/or therapeutic), i.e. indications for the use of rifampicin, include tuberculosis, meningococcal meningitis, leprosy, Haemophilus influenza, brucellosis, legionnaires disease, and serious staphylococcal infections.

In one embodiment, rifampicin is indicated for use in the treatment of all forms of tuberculosis, including fresh, advanced, chronic and drug-resistant cases, albeit in combination with other active anti-tuberculosis drugs. Rifampicin is also effective against most atypical strains of Mycobacteria.

In one embodiment, rifampicin is indicated for use in the treatment of meningococcal meningitis, in particular in the prophylaxis of meningococcal meningitis, for the treatment of asymptomatic carriers of N. meningitides, e.g. in the nasopharynx.

In one embodiment, rifampicin is indicated for use in the treatment of infections by Haemophilus influenzae, in particular for the treatment of asymptomatic carriers of H. influenza.

In one embodiment, rifampicin is indicated for use in the treatment of Leprosy, typically in combination with at least one other active anti-leprosy drug in the management of multibacillary and paucibacillary leprosy to effect conversion of the infectious state to a non-infectious state.

Patient in need of rifampicin treatment is a patient who would benefit from administration of rifampicin. Such patient may be suffering from any disease or condition for which rifampicin treatment may be useful in ameliorating symptoms. Such diseases or conditions include those caused by infections of organisms sensitive to rifampicin, such as the rifampicin indication.

As used herein, the term “avoid” and forms thereof are contemplated to have as alternatives the terms abstain, desist, forbear, and refrain, and forms thereof.

In a particular embodiment, the term “avoiding the use or administration of rifampicin” comprises or relates to avoidance of the use of rifampicin by looking into alternatives for the use of rifampicin in a patient in need of rifampicin therapy. Similarly, this may be the case for any other P-gp inducer contemplated for use in a patient in need of compound 1 therapy.

As used herein, the term “discontinue” and forms thereof, are contemplated to have as alternatives the terms cease, stop, suspend, and quit, and forms thereof.

As used herein, the term “contra-indicating” and forms thereof such as “contraindication” are contemplated to contain the instruction to not enter in the contraindicated activity.

Described herein (see example 1) is a human clinical study on the impact of rifampicin co-administration on the systemic exposure of compound 1. Rifampicin is a CYP3A4 and P-glycoprotein inducer. The study revealed an impact on both rate and extent of absorption, thereby substantially reducing the bio-availability of compound 1. Accordingly, aspects and embodiments are provided relating to avoidance, contraindication and/or discontinuation of use of CYP3A4/P-gp inducers in patients in need of compound 1 treatment. In embodiments described herein, in a patient in need of compound 1 treatment, e.g. a patient using or being administered compound 1, concomitant use or co-administration of CYP3A4/P-gp inducers, such as rifampicin, is avoided or contraindicated, or, administration of CYP3A4/P-gp inducer, such as rifampicin, is discontinued, to avoid adverse drug interaction, e.g. to avoid reduced systemic exposure of compound 1, potentially leading to and therefore also to avoid reduced (therapeutic) efficacy of compound 1 in said patient.

In some aspects is provided a method of administering compound 1 to a patient in need of compound 1 therapy, involving administering to the patient a therapeutically effective amount of compound 1, and avoiding use or administration (e.g. concomitant use or co-administration) of a CYP3A4/P-gp inducer in the patient also in need of treatment with said CYP3A4/P-gp inducer. In an embodiment is provided said method of administering compound 1 to a patient in need of compound 1 therapy, involving administering to the patient a therapeutically effective amount of compound 1, and avoiding use or administration (e.g. concomitant use or co-administration) of rifampicin. In a further embodiment, use or administration of the CYP3A4/P-gp inducer, such as rifampicin, is avoided by choosing an alternative treatment for the use or administration of the CYP3A4/P-gp inducer, such as rifampicin.

In some aspects is provided a method of administering compound 1 to a patient in need of compound 1 therapy, involving administering to the patient a therapeutically effective amount of compound 1, and discontinuing use or administration (e.g. concomitant use or co-administration) of a CYP3A4/P-gp inducer in the patient also in need of treatment with said CYP3A4/P-gp inducer. In an embodiment is provided said method of administering compound 1 to a patient in need of compound 1 therapy, involving administering to the patient a therapeutically effective amount of compound 1, and discontinuing the use or administration (e.g. concomitant use or co-administration) of rifampicin.

In some embodiments, the use or administration of the CYP3A4/P-gp inducer, such as rifampicin, is discontinued concurrent with starting administration or initiating use of compound 1. In other embodiments, the CYP3A4/P-gp inducer, such as rifampicin, is discontinued at least 1 day, at least 2 days or at least 3 days prior to starting use or administration of compound 1. In further embodiments, the CYP3A4/P-gp inducer, such as rifampicin, is discontinued at least 1 week, at least two weeks or at least three weeks prior to starting use or administration of compound 1.

In another embodiment, the CYP3A4/P-gp inducer, such as rifampicin, is discontinued within 1 day, within 2 days or within 3 days after starting use or administration of compound 1. These time periods can provide adequate time for tapering and withdrawal of the CYP3A4/P-gp inducer without adverse effects, e.g. when the CYP3A4/P-gp inducer is dexamethasone. The time after which use of a CYP3A4/P-gp inducer is discontinued should therefore be in line with safe withdrawal of the CYP3A4/P-gp inducer. In some embodiments, the CYP3A4/P-gp inducer is discontinued no later than one month, 3 weeks, 2 weeks or 1 week after starting compound 1 therapy.

In some aspects is provided a method of administering compound 1 to a patient in need of compound 1 therapy, involving administering to the patient a therapeutically effective amount of compound 1, and contraindicating use or administration (e.g. concomitant use or co-administration) of a CYP3A4/P-gp inducer in the patient also in need of treatment with said CYP3A4/P-gp inducer. In an embodiment is provided said method of administering compound 1 to a patient in need of compound 1 therapy, involving administering to the patient a therapeutically effective amount of compound 1, and contraindicating the use or administration (e.g. concomitant use or co-administration) of rifampicin.

In another aspect, a package or kit is provided comprising compound 1, optionally in a container, and, a package insert, package label, instructions or other labelling including information, recommendation or instruction regarding the avoidance or discontinuation or contraindication of concurrent use of compound 1 and rifampicin, or a CYP3A4/P-gp inducer in general, as described in different aspects and embodiments herein. E.g. such as a package insert, package label, instructions or other labelling may include any one or more of the following information, recommendation or instruction:
Informing or advising the patient that concurrent use of a CYP3A4/P-gp inducer, e.g. rifampicin, should be avoided;
Informing or advising the patient that concurrent use of a CYP3A4/P-gp inducer, e.g. rifampicin, is contraindicated;
Informing or advising the patient that concurrent use of a CYP3A4/P-gp inducer, e.g. rifampicin, should be discontinued, e.g. for at least 1 week, at least two weeks, at least 3 weeks or at least 4 weeks prior to compound 1 therapy;
Informing or advising the patient that concurrent use of a CYP3A4/P-gp inducer, e.g. rifampicin, can alter the therapeutic effect of compound 1, e.g. decreases the therapeutic effect of compound 1, in which case the instruction may further state that therefore concurrent use is contraindicated;
Instructing the patient to discontinue concurrent use of a CYP3A4/P-gp inducer, e.g. rifampicin;
Instructing the patient in need of compound 1 therapy to not use or administer concurrently a CYP3A4/P-gp inducer, e.g. rifampicin;
Contra-indicating the concurrent use or administration of a CYP3A4/P-gp inducer, e.g. rifampicin.

Instructions or recommendations may further include the advice to use alternative therapy replacing the use of the CYP3A4/P-gp inducer, e.g. rifampicin.

It is understood that the explicit wording included in a package insert, package label, instructions or other labelling may vary according to the jurisdiction where the package is authorized to be marketed and any wording that relates to the avoidance, discontinuation or contraindication of concurrent use or administration of a P-gp inducer, e.g. rifampicin, in a patient in need of compound 1 therapy, is hereby contemplated.

In a related aspect or embodiments is provided a method of preparing or packaging a compound 1 medicament comprising packaging compound 1, optionally in a container, together with a package insert, package label, instructions or other labelling including any one, two, three or more of the foregoing instructions, advise or recommendations.

### Example 1

A phase I, open-label, non-randomized, drug-drug interaction study in healthy male subjects is done to evaluate the impact of repeat doses of rifampicin, on the single-dose pharmacokinetics (PK) of compound 1 as well as to evaluate the safety and tolerability of a single dose of compound 1 administered before or after oral doses of rifampicin.

Eighteen healthy male subjects received a single oral dose of 600 mg compound 1 on Day 1. From Days 2 to 11, 600 mg rifampicin was administered once daily (q.d.) to all subjects for 10 consecutive days. On Day 12, subjects received a single oral dose of 600 mg of compound 1 (see Table 1). A follow-up visit is conducted between days 15 and 18.

TABLE 1
compound 1 and rifampin administration schedule

| Day 1 | Days 2 to 11 | Day 12 |
|---|---|---|
| Compound 1 | Rifampicin | Compound 1 |
| 600 mg | 600 mg q.d. | 600 mg |
| After standard | Fasted (min. 4 h), 30-60 min | After standard |
| breakfast | before food intake | breakfast |

### Samples for Determination of Compound 1

On days 1, 2, 12, 13 (i.e. 1, 2, 3, 4, 5, 6, 7, 8, 12 and 24 hours post compound 1 administration on day 1 and day 12) and at the follow-up visit, blood samples for PK assessment of compound 1 are taken. Blood samples of 2 mL for the determination of compound 1 levels in plasma are collected by venipuncture (or indwelling catheter) in the arm, into vacuum tubes containing lithium heparin and are immediately chilled (ice bath).

Within 30 min after blood collection, the plasma is separated by centrifugation at 4° C. for 10 min at circa 1,500 g and transferred into 2 polypropylene tubes with at least 400 μL of plasma in the first aliquot and the remaining volume in the second aliquot. After appropriate labeling, the plasma samples are stored in the clinical center at −20° C.±10° C. within 2 h following centrifugation and are protected from direct sunlight.

### Pharmacokinetic Assessment

Plasma concentrations were measured using validated liquid chromatography with tandem mass (LC/MS-MS) spectrometry methods with a limit of quantification of 1.00 ng/mL for compound 1, 20.0 mg/mL for cholesterol and 5.00 ng/mL for 4-β-hydroxycholesterol. Non-compartmental PK calculations are performed using Phoenix WinNonlin® (version 6.4).

The following parameters are determined for compound 1 from individual concentration-time profiles in plasma:
Cₘₐₓ: maximum observed plasma concentration
tₘₐₓ: time of occurrence of Cₘₐₓ
AUC₀₋ₜ: area under the plasma concentration-time curve from time zero till the time corresponding with the last observed quantifiable concentration, calculated by the linear up-logarithmic down trapezoidal rule
AUC₀₋₂₄ₕ: area under the plasma concentration-time curve from time zero till 24 h post-dose, calculated by the linear up-logarithmic down trapezoidal rule
C₂₄ₕ: plasma concentration observed at 24 h post-dose

The induction of CYP3A4 by rifampin is assessed using the ratio of 4-β-hydroxycholesterol/cholesterol as a marker and proves to be effective.

### Pharmacokinetic Analysis

Plasma concentrations and PK parameters of compound 1 are listed by day (Day 1: compound 1 before multiple rifampin doses; Day 12: compound 1 after multiple rifampin doses) for each subject. Descriptive statistics are calculated by day for the plasma concentration and PK parameters. Mean (±SE) concentration-time profiles are generated by day.

### Assessment of Drug Interaction

A mixed-effects model with day as fixed effect and subject as random effect is performed on the following natural log (ln)-transformed PK parameters of compound 1: Cₘₐₓ, C₂₄ₕ, AUC₀₋ₜ, and AUC₀₋₂₄ₕ. Point estimates are calculated as the geometric mean of the individual ratios for each parameter for Day 1 relative to the Day 12 and expressed as a percentage. The 90% confidence interval (CI) of the point estimate is calculated using the mean square error of the analysis of variance.

As tₘₐₓ is a discrete variable dependent on selected blood sampling times, day effect was assessed using a non-parametric test (Wilcoxon's signed-rank test).

### Pharmacokinetic Results

### Compound 1 Plasma Concentrations

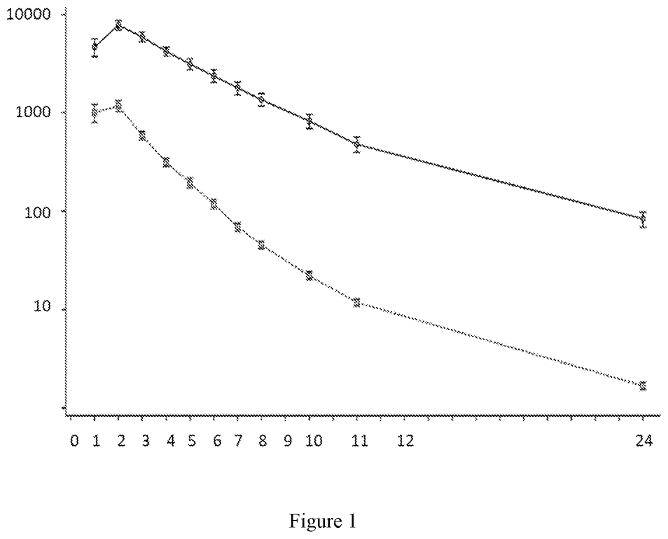

Mean (±SE) profiles are presented in FIG. 1 (log-linear scale).

Mean maximal compound 1 concentrations are attained at 2 h post-dose on Days 1 and 12 with values of 7,870 ng/mL and 1,180 ng/mL, respectively.

The elimination of compound 1 appears biphasic before and after multiple rifampin intake (FIG. 1). Visual inspection of the log-linear mean concentration-time profiles suggests marginal difference on the apparent rate of elimination when comparing Day 12 and Day 1, indicating no impact on compound 1 rate of elimination, and therefore no impact on compound 1 clearance.

### Compound 1 Pharmacokinetic Parameters

Individual compound 1 PK parameters with descriptive statistics and statistical analysis results of drug-drug interaction assessment are summarized in Table 2.

TABLE 2
Summary of compound 1 Pharmacokinetic Parameters and
Statistical Analysis of Drug-Drug Interaction Assessment

| | compound 1 | compound 1 | Comparison |
|---|---|---|---|
| | before multiple | after multiple | (PE % |
| | rifampin doses | rifampin doses | [90% CI] or |
| | N = 18 | N = 18 | p-value)^{a} |
| Cₘₐₓ | 8.66 | 1.44 | 16.78 |
| (μg/mL) | (46.7) | (53.8) | [13.33, 21.12] |
| C₂₄ₕ | 0.0834 | 0.00169 | 2.48 |
| (μg/mL) | (74.6) | (40.7) | [1.93, 3.18] |
| tₘₐₓ (h) | 2.00 | 2.00 | 0.7226 |
| | (1.00, 5.00) | (1.00, 6.00) | |
| AUC₀₋ₜ | 36.3 | 3.57 | 10.24 |
| (μg · h/mL) | (46.8) | (48.2) | [8.25, 12.72] |
| AUC₀₋₂₄ₕ | 36.3 | 3.75^{b} | 10.75 |
| (μg · h/mL) | (46.8) | (42.4) | [8.54, 13.54] |

PE = point estimate
N = number of subjects with data
For Cₘₐₓ, C₂₄ₕ, AUC₀₋ₜ and AUC₀₋₂₄ₕ, arithmetic mean (CV %) is presented. For tₘₐₓ median (mm, max) is presented
^{a}PE% [90% CI] presented for Cₘₐₓ, C₂₄ₕ, AUC₀₋ₜ and AUC₀₋₂₄ₕ, being the point estimate of the least-squares geometric means ratio of compound 1 at Day 12 relative to Day 1, and p-value presented for tₘₐₓ
^{b}N = 17 as 1 subject had evaluable data only up to 12 hours

The statistical analysis shows a significant day effect for Cₘₐₓ and AUCs while no significant effect is observed for tₘₐₓ (p-value 0.7226).

As shown by the point estimates (PEs), multiple rifampin intake results in a markedly lower bioavailability of compound 1: Cₘₐₓ and AUC₀₋₂₄ₕ are significantly decreased by respectively 6.0- and 9.3-fold (83.2 and 89.2%). The absence of impact on tₘₐₓ suggests an impact on the rate of absorption.

The between-subject variability in compound 1 PK parameters (Cₘₐₓ and AUCs), is moderate to high with CV % ranging between 46.7% and 53.8%.

The objective of the present study is to investigate the effect of multiple oral doses of rifampin on the single dose PK of compound 1.

After a single 600 mg oral dose, maximum compound 1 plasma concentration is reached rapidly (2 h) with an associated mean value of 7,870 ng/mL.

Once daily dosing with rifampin (600 mg q.d.) for 10 days leads to a decrease in bioavailability of compound 1 (by 6.0 and 9.3-fold for Cₘₐₓ and AUC₀₋₂₄ₕ, respectively), while no significant impact on tₘₐₓ is observed. Comparison of the plasma profiles shows no impact on the apparent rate of elimination of compound 1.

1. Compound 1 for use in treating a patient in need of compound 1 therapy, characterized in that the treating comprises avoiding, contraindicating or discontinuing concomitant use or co-administration of a combined CYP3A4/P-gp inducer.

2. The use of compound 1 in the manufacture of a medicament for treating a patient in need of therapy using compound 1, characterized in that the treating comprises avoiding, contraindicating or discontinuing concomitant use or co-administration of a combined CYP3A4/P-gp inducer.

3. A method of administering treatment using compound 1 to a patient in need of therapy using compound 1 comprising administering the patient a therapeutically effective amount of compound 1, and, avoiding, contraindicating or discontinuing concomitant use or co-administration of a combined CYP3A4/P-gp inducer.

4. Compound 1 for use, use of compound 1 or method according to any of the preceding claims wherein the patient in need of therapy using compound 1 is in need of administration of or therapy using the combined CYP3A4/P-gp inducer.

5. Compound 1 for use, use of compound 1 or method according to any of the preceding claims wherein the use or method comprises the step of discontinuing the use of a combined CYP3A4/P-gp inducer prior to or at the same time as the step of starting compound 1 therapy.

6. Compound 1 for use, use of compound 1 or method according to any of the preceding claims wherein the combined CYP3A4/P-gp inducer is rifampicin.

7. Compound 1 for use, use of compound 1 or method according to claim 6 wherein the use or method further comprises the step of replacing the rifampicin treatment by an alternative treatment.

8. Compound 1 for use, use of compound 1 or method according to any of the preceding claims wherein the patient in need of therapy using compound 1 is in need of rifampicin therapy.

9. Compound 1 for use, use of compound 1 or method according to any of the preceding claims wherein the patient in need of compound 1 therapy has pulmonary fibrosis, such as idiopathic pulmonary fibrosis.

10. Compound 1 for use, use of compound 1 or method according to any of the preceding claims wherein the patient has active or latent TB.

11. Compound 1 for use, use of compound 1 or method according to any of the preceding claims wherein compound 1 is administered at a daily dose of at least 100 or at least 200 mg.

12. Compound 1 for use, use of compound 1 or method according to any of the preceding claims wherein compound 1 is administered at a daily dose of 200 to 600 mg.

13. A package or kit comprising (i) compound 1, and (ii) a package insert, package label, instructions or other labelling comprising instructions to avoid or discontinue, or, contraindication of concomitant use or co-administration of a combined CYP3A4/P-gp inducer, in particular rifampicin.

14. A package or kit according to claim 14 further comprising one or more of the features according to claims 1 to 13.
